(19) United States
(12) Patent Application Publication
(10) Pub. No.: US 2008/0294119 A1
(43) Pub. Date: Nov. 27, 2008

(54) CATHETER POSITIONING APPARATUS

(75) Inventors: Steven N. Schwartz, Chesterfield, MO (US); Deborah L. Bauer, Millstadt, IL (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: Cath-A-Syst Enterprises, LLC

(21) Appl. No.: 11/937,786

(22) Filed: Nov. 9, 2007

Related U.S. Application Data
(60) Provisional application No. 60/857,990, filed on Nov. 10, 2006.

(51) Int. Cl. A61M 25/02; A61F 13/15; A61M 25/18

(52) U.S. Cl. 604/180; 604/358; 604/533

(57) ABSTRACT

Positioning apparatus is provided for positioning a catheter or similar medical or veterinary article relative to a patient's body. A tubular connector piece attaches an indwelling catheter to medical tubing or an injection port. The connector piece securely anchors the interconnected components to one another via leuer lock devices, and to a patient's skin through the use of a pre-attached adhesive-coated pad.

### RELATED APPLICATION

This application claims priority from U.S. Provisional Application No. 60/857,990, filed Nov. 10, 2006, the subject matter of which is incorporated herein by reference.

### BACKGROUND OF THE INVENTION

1. Field of the Invention

The present invention relates in general to a medical or veterinary article positioning apparatus for use with a human or animal patent. In one application, the present invention involves a catheterization system that interconnects and reversibly locks an in dwelling catheter to medical tubing or an injection cap, and securely anchors them to a patient's skin.

2. Description of the Related Art

Medical treatment of human and veterinary patients may involve the use of percutaneously inserted catheters to deliver fluids directly into the bloodstream, a specific organ or an internal location within the patient, or to monitor vital functions of the patient. As an example, peripherally-inserted, intravenous catheters are commonly used to administer fluids and/or medications directly into the bloodstream of the patient.

During intravenous fluid administration, the fluid typically drains from a container positioned above the patient and is delivered by gravity or via an infusion pump. The fluid flows through tubing and then into the indwelling catheter. The catheter and the fluid tubing are commonly removably attached to each other by a conventional leuer lock connection. Likewise, direct injections of medication into the intravenous catheter may be performed through an injection cap or syringe either of which commonly affixes to the catheter hub via a leuer lock mechanism.

In both human and veterinary medical practices surgical tape may be used to stabilize the catheter to the limb upon its initial insertion. Veterinary applications require that the healthcare provider first wrap thin strips of surgical tape 360 degrees around the base of the catheter hub immediately after placement into the vein, and then further secure the catheter by running the same strips of tape circumferentially around the patient's limb. This is intended to provide initial temporary stabilization of the catheter. The veterinary healthcare provider then places additional pieces of tape and/or other bandage material over the catheter hub, as well as over a safety loop in the intravenous tubing so that any tension applied to the tubing does not directly pass to the catheter. The use of surgical tape in the catheter stabilization process can be quite cumbersome and inefficient in both human and veterinary medical practice, but especially so during veterinary applications. This often occurs as a result of the uncooperative nature of the veterinary patient compounding the preexisting tendencies for excessive motion at the catheter insertion site, and for the thin strips of surgical tape to stick to themselves, to the health care provider's hands, or to the patient's hair adjacent to the catheter insertion site before the device is secured.

The entire taping and bandaging procedure takes several minutes of the healthcare provider's valuable time. In veterinary medical applications time constraints are most critical since the lack of patient cooperation dictates that the placement and securing of the catheter be performed as rapidly as possible to prevent catheter migration and/or dislodgment. Intravenous catheters are frequently pulled out during “routine” veterinary placement and attachment with surgical tape, even when performed by experienced healthcare providers.

In current human and veterinary medical applications, the catheter hub is attached directly to an injection port or intravenous fluid line. Connection sites require extreme security between the components of the catheter and fluid/medicine delivery system. This is often accomplished using a leuer lock connection which generally includes a male leuer connector with a tapered conical portion that is adapted to fit into a correspondingly shaped receptacle of a female leuer connector (i.e., a hub). A spin nut is commonly present on the male leuer connector and is rotatable relative to the tapered conical portion. The spin nut includes internal threads that are adapted to engage external threads on the female leuer connector to lock together the leuer connectors. When properly engaged, the conical portion fits tightly within the receptacle to produce a sealed interconnection.

The conical shape of the spin nut coupled with the size of its diameter result in lifting of the catheter hub up off of the skin surface when the devices are interlocked. Subsequent tension on the catheter may lead to partial or full obstruction of the catheter lumen, and increases the likelihood of migration and/or dislodgment. Currently, various stabilizers employed in human medical practice address this tendency by providing some degree of slope within a separate grasping mechanism (e.g. U.S. Pat. No. 6,290,676). In addition a number of retaining devices utilize adhesive coated pads which extend from the catheter grasping mechanism, and afford increased security of attachment. The bulkiness of such attachment systems precludes their use in veterinary applications where the patient's limb size may become a limiting factor.

Catheter insertion often results in contamination of the site with varying amounts of the patient's blood. Any such leakage must be cleaned as expeditiously as possible. In human medicine, direct exposure to this potentially infectious material puts the health care professionally at great risk. In contrast, blood contamination in veterinary applications interferes with the process of catheter stabilization itself, and is rarely of zoonotic significance. Contact with the animal's blood may occur when attachment of the injection port or IV tubing is delayed while the veterinary health care professional confirms backwards bloodflow into the catheter hub. The presence of even small amounts of residual blood at the site of catheter insertion precludes adhesion of the tape to both the catheter and skin. Any such blood contamination must first be removed using gauze pads or similar materials prior to taping and affixing the catheter. Until this point in the veterinary catheterization procedure, the catheter remains totally unsecured and vulnerable to dislodgement.

A variety of materials and devices are utilized to obtain maximum security between the components of the intravenous delivery system and the limb itself. In veterinary applications, additional layers of tape and/or other bandage material are placed over the catheter after it's initially affixed to the limb. These articles are first torn or cut with scissors into strips of various length and width, and then applied. This process may be quite awkward as the strips of material may stick to themselves, or to the working surface upon which they are placed. In addition, they may become moist from exposure to intravenous fluids, blood, or liquids used in cleansing and disinfection of the catheterization site resulting in a substantial decrease in their adhesive properties.

### SUMMARY OF THE INVENTION

The present invention provides a new and improved positioning apparatus for use in positioning a catheter or other device relative to a patient's body. The apparatus includes a base having a central portion which is to be connected with the catheter. In addition, the base has first and second sections which extend from the central portion of the base. Strips of adhesive are disposed on the sections of the base. Separator strips engage the strips of adhesive when the separator strips are in a blocking condition. The separator strips are movable to a disengaged condition in which they are separated from the strips of adhesive to enable the strips of adhesive to adhere to the patient's body. Absorbent pads are disposed on the separator strips and face away from the adhesive to facilitate absorption of any liquid disposed on the patient's body at a location to be engaged by a strip of adhesive.

A connector assembly is provided to interconnect the catheter and a conduit. The connector assembly includes a nut which engages the catheter. A positioning device may be utilized to orient the nut so that a pre-selected surface area on the nut faces toward the patient's body.

Although the present invention has a plurality of different features which are disclosed herein as being utilized together, it is contemplated that each of the features may be utilized separately from other features of the invention. It is also contemplated that each of the features of the invention may be utilized in combination with features from the prior art. It is also contemplated that the various features of the invention may be used with patients which are either humans or animals.

### BRIEF DESCRIPTION OF THE DRAWINGS

The foregoing and other features of the invention will become more apparent upon a consideration of the following description taken in connection with the accompanying drawings wherein:

FIG. 1 is a schematic illustration depicting the manner in which a positioning apparatus constructed in accordance with the present invention is utilized to position a catheter and a conduit relative to a patient's body;

FIG. 2 is a plan view of the positioning apparatus of FIG. 1, the catheter and conduit having been disconnected from the apparatus;

FIG. 3 is an elevational view, taken generally along the lines of 3-3 of FIG. 2, further illustrating the construction of the positioning apparatus;

FIG. 4 is an enlarged fragmentary schematic illustration of a portion of the positioning apparatus of FIG. 3 and depicting the relationship between an adhesive strip on a base section, a separator strip, and an absorbent pad on the separator strip;

FIG. 5 is an enlarged schematic cross sectional view illustrating a flat side surface formed on a leuer lock nut of a connector assembly which forms part of the positioning apparatus of FIGS. 2 and 3;

FIG. 6 is a schematic illustration, generally similar to FIG. 1, depicting the manner in which the positioning apparatus of FIGS. 1-3 may be enclosed by a flexible strip of material;

FIG. 7 is an enlarged fragmentary schematic illustration, generally similar to FIG. 4, illustrating an embodiment of the invention in which a second absorbent pad is disposed on a separator strip;

FIG. 8 is a plan view, generally similar to FIG. 2, illustrating an embodiment of the positioning apparatus having base sections which are skewed at an acute angle to a longitudinal central axis of the positioning apparatus;

FIG. 9 is a plan view, generally similar to FIG. 8, illustrating an embodiment of the positioning apparatus having base sections which are disposed in different angular relationships to a longitudinal central axis of the positioning apparatus;

FIG. 10 is an enlarged, schematic sectional view, similar to FIG. 5, illustrating an embodiment of the apparatus in which a leuer lock nut in a connector assembly cooperates with a positioning device which orients a side surface area on the nut relative to the patient's body;

FIG. 11 is a schematic sectional view illustrating the relationship between the nut of FIG. 10 and a conduit prior to the connection of a catheter with the conduit;

FIG. 12 is a schematic sectional view, generally similar to FIG. 11, illustrating the relationship between the conduit, nut and catheter after the catheter has been connected with the conduit by the nut; and

FIG. 13 is a schematic illustration of an embodiment of the positioning apparatus having an auxiliary fluid conduit connected a main fluid conduit.

### DESCRIPTION OF SPECIFIC PREFERRED EMBODIMENTS OF THE INVENTION

### General Description

A positioning apparatus 10, constructed in accordance with the present invention, is illustrated in FIG. 1 in association with a portion 12 of a patient's body. The positioning apparatus 10 is utilized to position a catheter 14 and a fluid conduit 16 relative to the portion 12 of the patient's body. However, the positioning apparatus 10 may be used to position other known devices relative to a patient's body. The portion 12 of the patient's body may be a leg, arm or other part of the patient's body. The patient may be either a human or an animal.

The positioning apparatus 10 includes a connector assembly 22 which connects the fluid conduit 16 in fluid communication with the catheter 14. The fluid connection provided between the conduit 16 and catheter 14 by the connector assembly 22 enables fluid to be conducted between the conduit and the catheter. If desired, a syringe may be connected with the catheter 14 by the connector assembly 22 in place of the conduit 16. If this is done, the needle or the needle hub of the syringe would form a conduit corresponding to the conduit 16.

During intravenous fluid administration, the conduit 16 may be connected with a reservoir of fluid disposed at a level above the catheter 14 and portion 12 of the patient's body. This results in fluid being conducted, under the influence of gravity, through the fluid conduit 16, connector assembly 22 and catheter 14 into the portion 12 of the patient's body. Alternatively, the fluid conduit 16 may be connected with an infusion pump. This would result in fluid being conducted from the pump through the conduit 16, connector assembly 22 and catheter 14 into the portion 12 of the patient's body. If the conduit 16 is a needle or the needle hub of a syringe, the syringe may be used to either supply fluid to the patient or to withdraw fluid from the patient.

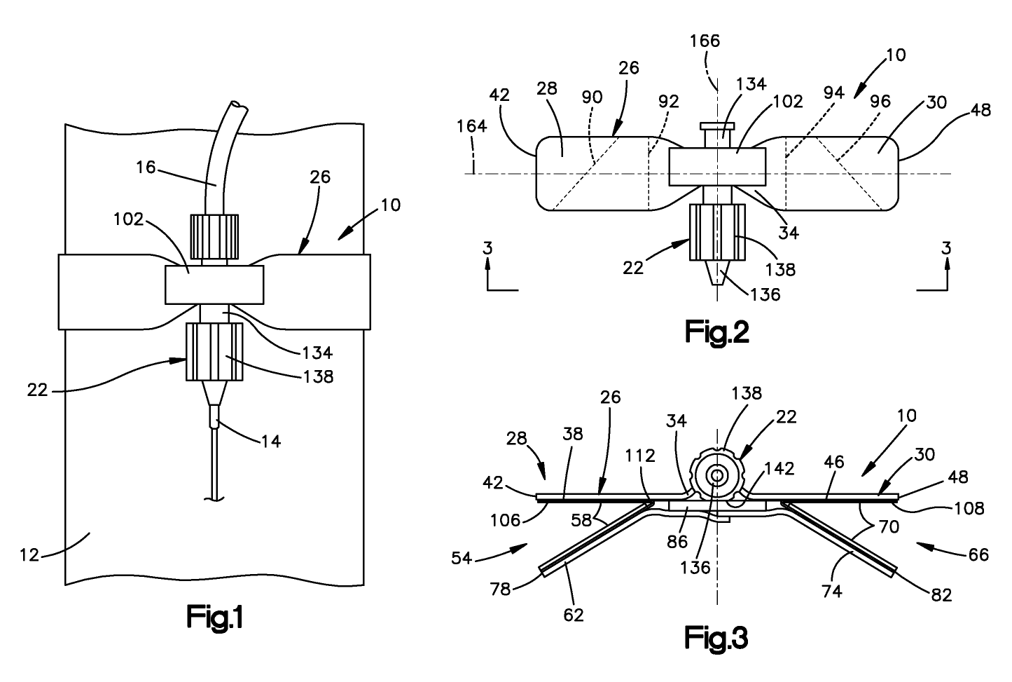

Regardless of whether or not a syringe is utilized to provide the conduit 16, the positioning apparatus 10 is utilized to grip the connector assembly 22 and to anchor the connector assembly against movement relative to the patient's body. The positioning apparatus 10 includes a flexible base 26 having left and right (as viewed in FIGS. 2 and 3) sections 28 and 30. The left and right sections 28 and 30 of the base 26 are interconnected by a central portion 34 of the base.

The central portion 34 of the base 26 is integrally formed as one piece with the left and right sections 28 and 30 and extends across an upper (as viewed in FIG. 3) side of the connector assembly 22. However, if desired, the base 26 may be formed by a plurality of interconnected pieces. For example, the left and right sections 28 and 30 may be formed separately from the central portion 34.

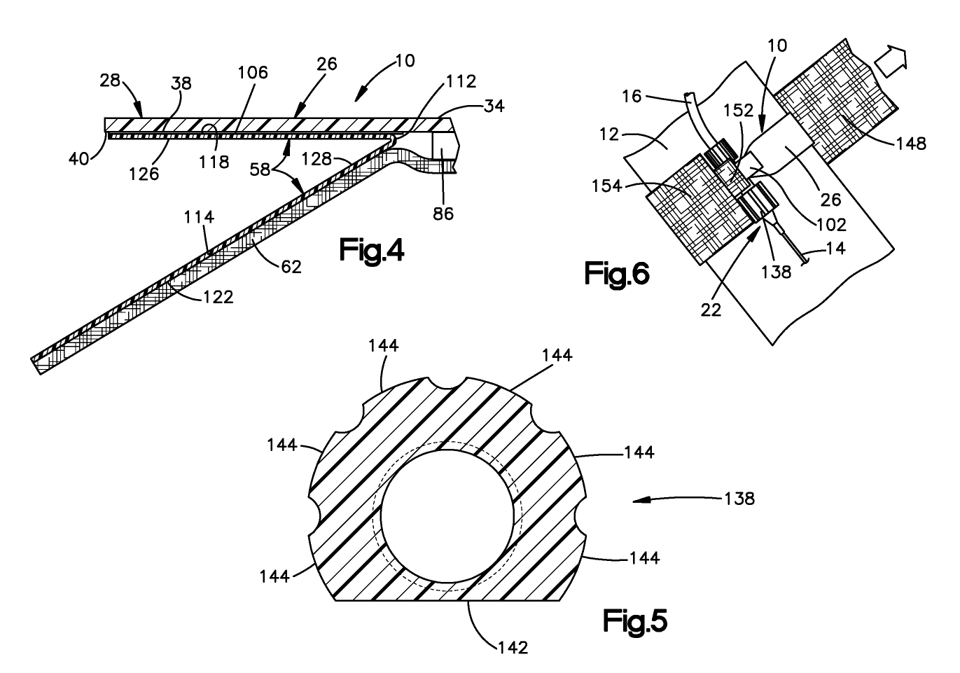

To enable the left section 28 of the flexible base 26 to firmly grip the skin on the portion 12 of the patient's body, a left strip 38 (FIGS. 3 and 4) of adhesive is provided on a downwardly (as viewed in FIG. 4) facing side surface 40 of the left section 28 of the base 26. The left strip 38 of adhesive extends outwardly from the central portion 34 of the base 26 to a tip or end portion 42 (FIG. 3) of the left section of the base.

Similarly, to enable the right section 30 (FIGS. 2 and 3) of the flexible base 26 to firmly grip the skin on the portion 12 (FIG. 1) of the patient's body, a right adhesive strip 46 is provided on a downwardly (as viewed in FIG. 3) facing side surface of the right section 30 of the base 26. The right adhesive strip 46 extends outwardly from the central portion 34 of the base 26 to a tip or end portion 48 of the right section 30 of the base 26.

The central portion 34 of the base 26 may have a strip of adhesive to connect the base with the connector assembly 22. The strip of adhesive on the central portion 34 of the base 26 may be integrally formed with the left and right strips 38 and 46 to form one continuous adhesive strip. Alternatively, the strip of adhesive on the central portion 34 of the base 26 may be spaced from and disposed between the left and right strips 38 and 46 of adhesive

When the left and right strips 38 and 46 of adhesive are firmly pressed against the skin on the portion 12 of the patient's body, the positioning apparatus 10 (FIG. 1) is secured to the patient's body. This results in the connector assembly 22 being held against movement relative to the portion 12 of the patient's body. Of course, holding the connector assembly 22 against movement relative to the portion 12 of the patient's body is also effective to hold the catheter 14 against movement relative to the portion 12 of the patient's body when catheter 14 is engaged by the connector assembly 22. In addition, the lower (as viewed in FIG. 1) end portion of the fluid conduit 16, when engaged by the corresponding attachment site of positioning apparatus 10, is held against movement relative to the portion 12 of the patient's body by adherence of the left and right strips 38 and 46 (FIG. 3) of adhesive to the skin on the patient's body.

If a syringe is to be utilized in association with the catheter 14, the positioning apparatus 10 holds the connector assembly 22 and catheter against movement relative to the portion 12 of the patient's body during connection and/or disconnection of the syringe from the connector assembly. When a syringe is utilized, an injection plug may be associated with the connector assembly 22 and catheter 14 to form a port where the needle of the syringe is inserted.

A left detachable portion 54 (FIGS. 3 and 4) is connected with the left section 28 of the base 26 by the left strip 38 of adhesive. The left detachable portion 54 protects the left strip 38 of adhesive prior to positioning of the left strip of adhesive relative to the portion 12 of the patient's body. In addition, the left detachable portion 54 may be utilized to absorb any liquid, such as perspiration and/or blood, which may be on the patient's skin prior to engagement of the left strip 38 of adhesive with the patient's skin.

The left detachable portion 54 includes a separator strip 58. The separator strip 58 is formed as one piece and has a generally V-shaped cross sectional configuration. The separator strip 58 may have a different configuration if desired. For example the separator strip 58 may be formed by a single layer. The separator strip 58 extends across the left strip 38 of adhesive to block unwanted engagement of the left strip 38 of adhesive with a patient and/or an environment around the patient.

An absorbent pad 62 is disposed on the separator strip 58 and faces away from the left strip 38 of adhesive. The absorbent pad 62 is connected to a portion of the separator strip 58 which is spaced from the left strip 38 of adhesive. The absorbent pad 62 may be connected to the separator strip 58 by a suitable adhesive or other connector. This enables the absorbent pad 62 to be moved, relative to the base 26, while the separator strip 58 is in engagement with the left strip 38 of adhesive.

An individual who is going to secure the positioning apparatus 10 (FIG. 1) to a portion 12 of a patient's body can move the absorbent pad 62 along the skin on the patient's body while removing the separator strip 58 to absorb any liquid which may be present. Thus, the absorbent pad 62 may be wiped along the patient's skin to absorb any perspiration and/or blood which may be present on the skin of the patient's body at a location where the positioning apparatus 10 is to be mounted.

A right detachable portion 66 (FIG. 3) is connected with the right section 30 of the base 26 by the right adhesive strip 46. The right detachable portion 66 protects the right strip 46 of adhesive prior to positioning of the right strip of adhesive relative to the portion 12 of the patient's body. In addition, the right detachable portion 66 may be utilized to absorb any liquid, such as perspiration and/or blood, which may be on the patient's skin prior to engagement of the right strip 46 of adhesive with the patient's skin.

The right detachable portion 66 includes a right separator strip 70. The separator strip 70 is formed as one piece and has a generally V-shaped cross section configuration. The separator strip 70 extends across the right strip 46 of adhesive to block unwanted engagement of the right strip 46 of adhesive with a patient and/or an environment around the patient. An absorbent pad 74 is disposed on the right separator strip 70 and faces away from the right strip 46 of adhesive. The absorbent pad 74 may be connected to the separator strip 70 by a suitable adhesive or other connector.

An individual who is going to secure the positioning apparatus 10 (FIG. 1) to a portion of a patient's body can move the absorbent pad 74 (FIG. 3) along the skin on the patient's body while removing the separator strip 70 to absorb any liquid which may be present. Thus, the absorbent pad 74 may be wiped along the patient's skin to absorb any perspiration and/or blood which may be present on the skin of the patient's body at a location where the positioning apparatus is to be mounted.

Upon insertion of the catheter 14 into the portion 12 of the patient's body and removal of an insertion needle from the catheter, there tends to be a backflow of blood which is deposited on the patient's skin. The connector assembly 22 is connected with the catheter 14 immediately after removal of the insertion needle. This minimizes the amount of blood which is deposited on the patient's skin.

The left and right absorbent pads 62 and 74 are wiped along the portion of the patient's skin on which the positioning apparatus 10 is to be mounted as separator strips 58 and 70 are removed to expose adhesive strips 38 and 46 respectively. This cleans the patient's skin and removes any blood or other foreign material which may be disposed on the patient's skin. Since the absorbent pads 62 and 74 are mounted on portions of the separator strips 58 and 70 which are movable relative to the base 26, wiping of the patient's skin with the absorbent pads is facilitated.

To remove the left separator strip 58, an end portion 78 (FIG. 3) of the left separator strip 58 and absorbent pad 62 are manually gripped and pulled toward the left (as viewed in FIG. 3). This results in the separator strip 58 moving away from the left strip 38 of adhesive to expose the left strip of adhesive. As the left separator strip 58 is being removed from the left strip 38 of adhesive, any blood on the patient's skin adjacent to the left section 28 of the base 26 is removed by being wiped away with the absorbent pad 62. The left section 28 of the base 26 may then be manually pressed against the patient's skin to secure the connector assembly 22 in place on the patient's skin.

To remove the right separator strip 70, an end portion 82 (FIG. 3) of the right separator strip 70 and right absorbent pad 74 are manually gripped and pulled toward the right (as viewed in FIG. 3). This disengages the separator strip 70 from the right strip 46 of adhesive to expose the right strip of adhesive. As the right separator strip 70 is being removed from the right strip 46 of adhesive, any blood on the patient's skin adjacent to the right section 30 of the base 26 is removed by being wiped away with the absorbent pad 74. The right section 30 of the base 26 is then positioned relative to the patient's body and the right strip 46 of adhesive pressed against the patient's body to secure the right section 30 of the base 26 in place on the patient's body. Of course, the right section 30 of the base 26 may be positioned on the portion 12 of the patient's body before the left section 28 of the base is positioned on the patient's body.

A non-absorbent pad 86 (FIG. 3) is connected to the central portion 34 of the base 26 by a suitable fastener, such as stitching or adhesive. If desired, the non-absorbent pad 86 may be connected with the base 26 by the left and right strips 38 and 46 of adhesive. The non-absorbent pad 86 is disposed between the connector assembly 22 and the patient's body to minimize irritation of the patient's body by the connector assembly. It should be understood that the non-absorbent pad 86 may be omitted if desired.

If desired, a non-absorbent support or cradle having an arcuate upper side surface may be positioned between the non-absorbent pad 86 and the connector assembly 22 to support the connector assembly. The arcuate side surface on the support engages a correspondingly shaped arcuate surface on the connector assembly. A flat side of the support cradle, facing away from the connector assembly 22, engages an upper (as viewed in FIG. 3) side of the non-absorbent pad 86. It should be understood that the support may be omitted if desired.

### Base

The left and right sections 28 and 30 and central portion 34 of the base 26 are integrally formed by a single piece of flexible material. Although the flexible material forming the left and right sections 28 and 30 and central portion 34 of the base 26 is a non woven synthetic fabric, it is contemplated that the flexible material forming the base 26 may be either a synthetic or non-synthetic material which is either woven or non-woven. Breathing holes are provided in the non-woven synthetic material forming the left and right sections 28 and 30 and central portion 34 of the base 26.

The material forming the base 26 is similar to the material commonly utilized in bandages. One specific embodiment of the base was formed of Nexcare (trademark) Comfort Bandage which is commercial available from 3M Corporation having a place of business at St. Paul, Minn. 55144. Of course other known materials may be used to form the base 26 if desired. For example, the base 26 may be formed by a sheet of polymeric material.

Although the illustrated base 26 is integrally formed of one piece of material, it is contemplated that the base 26 may be formed of a plurality of pieces of material. If this is done, a piece of material forming the left section 28 of the base 26 may be connected with a piece of material forming the right section 30 of the base. The piece of material which interconnects the left and right sections 28 and 30 of the base 26 may have a construction which is different than the construction of the material forming the left and right sections. For example, the non-absorbent pad 86 may interconnect separate pieces of material forming the left and right sections 28 and 30 of the base 26.

Fold lines 90, 92, 94 and 96 (FIG. 2) are formed in the flexible material forming the left and right sections 28 and 30 of the base 26 and in the left and right detachable portions 54 and 66. The fold lines 90-96 enable the positioning apparatus 10 to be compactly folded for packaging. Of course, if desired, the fold lines 90-96 may be omitted and the positioning apparatus 10 packaged in the extended configuration illustrated in FIGS. 2 and 3.

The central portion 34 of the base 26 extends across the upper side of the connector assembly 22, in the manner illustrated schematically in FIG. 2. A short strip 102 of fastener material is secured to the central portion 34 of the base 26 by adhesive and by stitching. Of course, the short strip 102 of fastener material may be secured to the base 26 in a different manner if desired.

The short strip 102 of fastener material is the hook portion of a hook and loop fastener (Velcro™). A short strip of loop material of a hook and loop fastener is utilized to connect an article to the strip 102 of fastener material. Although any desired article may be connected to the positioning apparatus 10 using the strip 102 of fastener material, a strip of flexible material for wrapping the positioning apparatus 10 and the portion 12 of the patient's body may advantageously be connected to the strip 102 of fastener material.

### Separator Strips

The left and right separator strips 58 and 70 have upper (as viewed in FIG. 3) portions 106 and 108 which are coextensive with the left and right strips 38 and 46 of adhesive on the left and right sections 28 and 30 of the base 26. The upper portions 106 and 108 of the separator strips 58 and 70 prevent adhesive strips 38 and 46 from being engaged by foreign materials prior to movement of the adhesive strips into engagement with the skin on a portion 12 of a patient's body. The left and right separator strips 58 and 70 are each formed of one piece of a polymeric material which is readily disengaged from the adhesive strip 38 or 46 by pulling on one end of the separator strip. Disengagement of the left and right separator strips 58 and 70 from the left and right adhesive strips 38 and 46 may be effected by pulling on the end portions 78 and 82 of the separator strips.

A fold 112 (FIG. 4) is formed in the left separator strip 58. The fold 112 extends across the left separator strip 58 and is perpendicular to a longitudinal central axis of the left separator strip. The fold 112 separates a lower (as viewed in FIG. 4) portion 114 of the left separator strip 58 from the upper portion 106 of the left separator strip. An upper (as viewed in FIG. 4) side surface 118 of the upper portion 106 of the separator strip 58 is disposed in engagement with the left strip 38 of adhesive throughout the extent of the left strip 38 of adhesive.

The lower (as viewed in FIG. 4) portion 114 of the separator strip 58 has a lower (as viewed in FIG. 4) side surface area 122. The absorbent pad 62 is secured to the lower side surface 122 of the left separator strip 58. The absorbent pad 62 extends from the end portion 78 (FIG. 3) to the fold 112 in the left separator strip 58. The upper side surface area 118 on the left separator strip 58 is formed as a continuation of the lower (as viewed in FIG. 4) side surface area 122 of the lower portion 114 of the separator strip. The upper side surface area 118 is separated from the lower side surface area 122 by the fold 112.

Prior to installation of the positioning apparatus 10 on a portion 12 of a patient's body, a lower side surface area 126 (FIG. 4) on the upper portion of the separator strip 106 is engaged by an upper (as viewed in FIG. 4) side surface area 128 on the lower portion 114 of the left separator strip 58. This results in the absorbent pad 62 being closely adjacent to the left strip of adhesive 38 and extending parallel to the left strip of adhesive. At this time, the absorbent pad 62 is separated from the left strip of adhesive by only the left separator strip 58. The absorbent pad 62 extends across the fold 112 in the separator strip 58 to the non-absorbent pad 86 (FIG. 3) which is connected to the central portion 34 of the base 26.

Although only the left separator strip 58 is illustrated in FIG. 4, it should be understood that the right separator strip 70 (FIG. 3) has the same construction as the left separator strip 58. The right separator strip 70 cooperates with the right strip 46 of adhesive and the absorbent pad 74 in the same manner as in which the left separator strip 58 cooperates with the left strip 38 of adhesive and the absorbent pad 62. The right detachable portion 66 has the same construction as the left detachable portion 54. Of course, the left and right detachable portions 54 and 66 may have a different construction if desired.

In the illustrated embodiment of the invention, each of the separator strips 58 and 70 has two layers interconnected by a fold. However, it is contemplated that each of the separator strips 58 and 70 may be formed by a single layer. If a single layer separator strip is used, the single layer of each separator strip would engage one of the adhesive strips 38 or 46. Each of the absorbent pads 62 and 74 would be disposed on a side of a separator strip opposite from one of the strips of adhesive engaged by the separator strip.

### Connector Assembly

The connector assembly 22 (FIG. 2) is mounted on the central portion 34 of the base and is effective to connect the catheter 14 in fluid communication with the conduit 16. Although a conduit 16 is connected with a source of fluid, such as a reservoir or pump, it should be understood that the conduit 16 may be a hypodermic needle which is connected with a syringe. The syringe may be utilized as a source of fluid for the catheter 14. Alternatively, the syringe may be utilized to withdraw fluid from the patient's body through the catheter 14 to the syringe.

The connector assembly 22 includes a tubular body 134 (FIG. 2) having a generally cylindrical configuration. The tubular body 134 is disposed between the pad 86 (FIG. 3) and the central portion 34 of the base 26. It may be desired to have the cylindrical tubular body 134 disposed in a recess in a support cradle positioned between the tubular body and the pad 86. Of course, the support cradle may be omitted if desired.

The tubular body 134 (FIG. 2) has a tapered leading end portion 136 which is formed as a frustrum of a right circular cone. The leading end portion 136 of the body 134 is telescopically inserted into a similarly tapered recess in the catheter 14. A leuer lock nut 138 is rotatably mounted on the tubular body 134 of the connector assembly 22. The leuer lock nut 138 has an internal thread convolution which engages a pair of flanges on an end portion of the catheter 14 to hold the tapered leading end portion 136 of the tubular body 134 in tight sealing engagement with a similarly configured recess in the catheter 14. The general construction of the connector assembly 22 is well-known and will not be further described herein to avoid prolixity of description.

To minimize raising of the catheter and the possibility of irritation of a patient's body by engagement with the leuer lock nut 138, the nut has a flat side surface 142 (FIGS. 3 and 5) which is disposed over the portion 12 of the patient's body when the nut is connected with a catheter in the manner illustrated in FIG. 1. When the leuer lock nut 138 is connected with the catheter 14, the flat side surface 142 (FIG. 5) on the nut is facing downward (as viewed in FIGS. 1-3) and lays flush with the patient's skin. This results in the end of the catheter 14 which is connected to the leuer lock nut 138 being very close to the patient's skin.

The leuer lock nut 138 has a plurality of arcuate side surfaces 144 (FIG. 5). The side surfaces 144 may be flat rather than having the arcuate configuration illustrated in FIG. 5. Regardless of the configuration of the side surfaces 144, the flat side surface 142 may have an extent, as viewed in a plane extending perpendicular to the longitudinal central axis of the leuer lock nut, which is greater than the extent of the side surfaces 144. The illustrated side surface 142 has an extent which is more than twice as great as the extents of the side surfaces 144. Of course, the surface 142 may be formed with a different extent if desired.

Although the illustrated lock nut 138 has a flat side surface 142, the flat side surface may be omitted if desired. Alternatively, the lock nut 138 may have a side surface area with a configuration which is not flat. For example, an arcuate concave surface area may be substituted for the flat side surface 142.

### Wrapping of the Positioning Apparatus

The positioning apparatus 10 (FIG. 1) may be enclosed with a protective strip 148 (FIG. 6) of fabric or other material. A short strip 152 of fastener material is connected to an end portion 154 of the protective strip 148. The strip 152 of fastener material engages the strip 102 (FIG. 2) of fastener material connected to the base 26 of the positioning apparatus 10. Since the strip 102 of fastener material connected to the base 26 of the positioning apparatus 10 is a hook type fastener material, the strip 152 of fastener material is a loop type of fastener material. Of course, the fastener material connected to the base 26 may be loop type fastener material and the strip 152 may be hook type. The hook and loop type fastener materials forming the strips 102 and 152 are similar to hook and loop type fasteners which are commercially available under the trademark Velcro™.

Once the strip 152 of fastener material on the end portion 154 of the fabric strip 148 has been secured to the strip 102 of fastener material on the base 26 of the positioning apparatus 10, the protective strip 148 is wrapped around the positioning apparatus 10. Although only a portion of a single turn of the protective strip 148 has been illustrated schematically in FIG. 6, it should be understood that a plurality of turns of the protective strip 148 are wrapped around the positioning apparatus 10 to fully enclose the positioning apparatus and the catheter 14. The free end of the protective strip 148, that is, the end opposite of the end portion 154, is self adhering to one of the turns of the protective strip. However, any desired type of known fastener may be utilized. For example, an adhesive may be utilized to connect the free end of the protective strip 148 with one of the turns of the protective strip.

The protective strip 148 is formed of a stretchable fabric. The fabric forming the protective strip has openings so that air can pass through the protective strip. It should be understood that the protective strip 148 may have a different construction if desired. For example, the protective strip 148 may be an adhesive bandage which adheres to the positioning apparatus 10 and to the portion 12 of the patient's body or it may be a non-adhesive, self-adherent material such as Coban™, VetWrap™, or PetFlex™.

### Alternative

### Detachable Portion

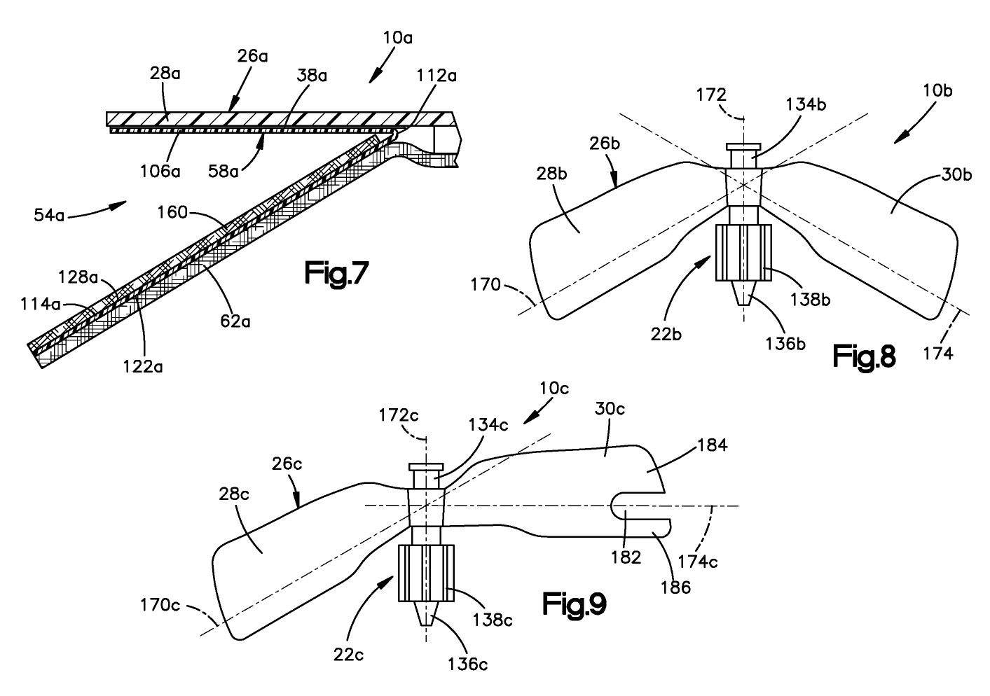

In the embodiment of the invention illustrated in FIGS. 3 and 4, an absorbent pad 62 is provided on one side of the separator strip 58. In the embodiment of the invention illustrated in FIG. 7, absorbent pads are provided on opposite sides of the separator strip. Since the embodiment of the invention illustrated in FIG. 7 is generally similar to the embodiment of the invention illustrated in FIGS. 1-6, similar numerals will be utilized to designate similar components, the suffix letter “a” being associated with the numerals of FIG. 7 to avoid confusion.

A left detachable portion 54a of a positioning apparatus 10a is illustrated in FIG. 7. The positioning apparatus 10a has the same construction and mode of operation as the positioning apparatus 10 of FIGS. 1-4. However, the positioning apparatus 10a of FIG. 7 has a pair of absorbent pads associated with a lower portion 114a of a separator strip 58a. The lower portion 114a of the separator strip 58a has a lower side surface 122a to which an absorbent pad 62a is connected by a suitable adhesive. The absorbent pad 62a extends past a fold 112a in the separator strip 58a. A second absorbent pad 160 is connected to an upper side surface area 128a on the lower portion 114a of the separator strip 58a. By providing a pair of absorbent pads 62a and 160 on the lower portion 114a of the separator strip 58a, wiping of a relatively large area on a patient's skin is facilitated.

In addition to the lower portion 114, the separator strip 58a includes an upper portion 106a which is integrally formed as one piece with the lower portion 114a and is connected with the lower portion 114a by the fold 112a in the separator strip 58a. The upper portion 106a of the separator strip 58a is connected to the left section 28a of the base 26a by a strip 38a of adhesive. If desired, the absorbent pad 160 may extend across the upper portion 106a of the separator strip 58a.

### Alternative

### Base Configurations

The base 26 (FIG. 2) includes left and right sections 28 and 30 having a common central axis 164 which extends perpendicular to a central axis 166 of the positioning apparatus 10. In the embodiment of the invention illustrated in FIG. 8, the base has left and right sections with central axes which are skewed relative to each other and to a central axis of the positioning apparatus. Since the embodiment of the invention illustrated in FIG. 8 is generally similar to the embodiments of FIGS. 1-6, similar numerals will be utilized to identify similar components, the suffix letter “b” being added to the numerals of FIG. 8 to avoid confusion.

A positioning apparatus 10b (FIG. 8) includes a connector assembly 22b which is connectable with a catheter and a conduit, similar to the catheter 14 and conduit 16 of FIG. 1. The positioning apparatus 10b includes a base 26b having left and right sections 28b and 30b. The connector assembly 22b includes a tubular body 134b having a tapered end portion 136b. A leuer lock nut 138b is rotatably mounted on the tubular body 134b and is disposed between the left and right base sections 28b and 30b.

The left base section 26b has a longitudinal central axis 170 which is skewed at an acute angle to a central axis 172 of the connector assembly 22b and of the positioning apparatus 10b. The right base section 30b has a central axis 174 which is skewed at an obtuse angle to the central axis 170 of the left section 28b. The central axis 174 of the right section 30b is skewed at an acute angle to the central axis 172 of the positioning apparatus 10b.

By having the central axes 170 and 174 of the left and right base sections 28b and 30b skewed at acute angles relative to the central axis 172 of the positioning apparatus 10b, the left and right strips of adhesive, corresponding to the strips 38 and 46 of FIG. 3, are engageable with portions of the patient's body which are offset along the longitudinal central axis 172 of the positioning apparatus 10b. It is believed that this will be particularly advantageous when the positioning apparatus 10b is utilized in association with an animal having hair or fur which must be shaved to provide access to the skin of the animal in order to secure the positioning apparatus 10b.

Fold lines, corresponding to the lines 90, 92, 94 and 96 (FIG. 2) have not been illustrated in association with the embodiment of FIG. 8. However, fold lines may be provided in association with the positioning apparatus 10b of FIG. 8. If fold lines, corresponding to the fold lines 90-96 of FIG. 2, are provided in association with the embodiment of FIG. 8, the fold lines may extend parallel to the central axis 172 of the positioning apparatus 10b. Alternatively, fold lines, corresponding to the fold lines 90-96 of FIG. 2, may extend perpendicular to the axes 170 and 174.

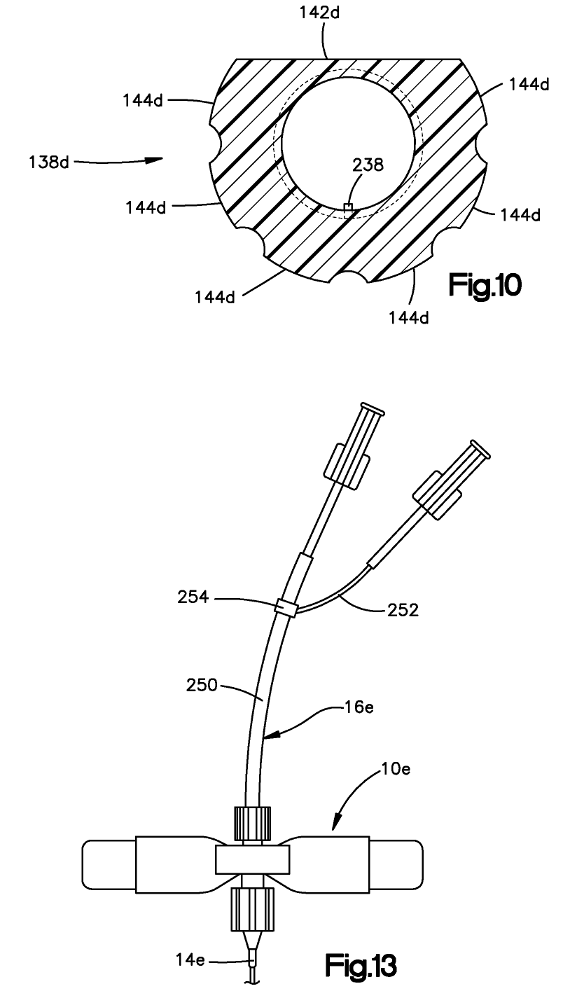

The base 26b of FIG. 8 includes left and right sections 28b and 30b having central axes 170 and 174 which are skewed at acute angles relative to a central axis 172 of the positioning apparatus 10b. In the embodiment of the invention illustrated in FIG. 9, only one of the sections of the base is skewed at an acute angle relative to the central axis of the positioning apparatus. Since the embodiment of the invention illustrated in FIG. 9 is generally similar to the embodiments of FIGS. 1-8, similar numerals will be utilized to identify similar components, the suffix letter “c” being added to the numerals of FIG. 9 to avoid confusion.

A positioning apparatus 10c (FIG. 9) includes a connector assembly 22c which is connectable with a catheter and a conduit, similar to the catheter 14 and conduit 16 of FIG. 1. The positioning apparatus 10c includes a base 26c having left and right sections 28c and 30c. The connector assembly 22c includes a tubular body 134c having a tapered end portion 136c. A leuer lock nut 138c is rotatably mounted on the tubular body 134c and is disposed between the left and right base sections 28c and 30c.

The left base section 28c has a longitudinal central axis 170c which is skewed at an acute angle to a central axis 172 of the connector assembly 22c and of the positioning apparatus 10c. The right base section 30c has a central axis 174c which is skewed at an obtuse angle to the central axis 170c of the left section 28c. The central axis 174c of the right section 30c is perpendicular to the central axis 172 of the positioning apparatus 10c.

By having the central axes 170c and 174c of the left and right base sections 28c and 30c skewed at different angles relative to the central axis 172c of the positioning apparatus 10c, the left and right strips of adhesive, corresponding to the strips 38 and 46 of FIG. 3, are engageable with portions of the patient's body which are offset along the longitudinal central axis 172 of the positioning apparatus 10c.

The right base section 30c is provided with a recess 182 (FIG. 9) which provides space to receive the strip 102 (FIG. 2) of fastener material. When the base section 30c is wrapped around the body portion 12 (FIG. 1), the recess 182 (FIG. 9) moves into alignment with the strip 102 of fastener material. This results in the strip 102 of fastener material being exposed at the recess 182. Portions 184 and 186 (FIG. 9) of the base section 30c are disposed on opposite sides of the strip 102 of fastener material. This enables the strip 152 (FIG. 6) of fastener material on the end portion of the protective strip 148 to engage the strip 102 of fastener material when the base section 30c when the base section has been wrapped around the portion 12 of the patient's body and overlies the connector assembly 22c.

Although a recess 182 has been illustrated in association with only the embodiment of FIG. 9, it should be understood that the recess 182 may be used with any of the embodiments of the invention illustrated herein. For example, a recess, corresponding to the recess 182, may be provided in the base section 30 of FIG. 2. If desired, a recess, corresponding to the recess 182 (FIG. 9) may be provide in both the base section 28 (FIG. 2) and the base section 30. This would enable the two base sections 28 and 30 to be disposed in an overlapping relationship with the connector assembly 22 while still providing access to the strip 102 of fastener material through the aligned recesses.

### Positioning Device

In the embodiment of the invention illustrated in FIGS. 1-6, the leuer lock nut 138 (FIG. 1) is provided with a flat side surface 142 (FIG. 5). When the internally threaded lock nut 138 is connected with an external thread on the catheter 14, the nut is rotated relative to the base 26 (FIG. 1) of the positioning apparatus 10. Depending upon the position in which the nut is located when it engages the external thread on the catheter 14, the flat side surface 142 may or may not move to the orientation illustrated in FIGS. 3 and 5 in which the flat side surface faces toward the patient's body portion 12.

In the embodiment of the invention illustrated in FIGS. 10, 11 and 12, a positioning device is provided to orient the leuer lock nut so that the flat surface area on the nut will always face toward the patient's body when the catheter 14 is connected with the tubular body 134. Since the embodiment of the invention illustrated in FIGS. 10-12 is generally similar to the embodiments of the invention illustrated in FIGS. 1-9, similar numerals will be utilized to designate similar components, the suffix letter “d” being associated with the numerals of FIGS. 10-12 to avoid confusion.

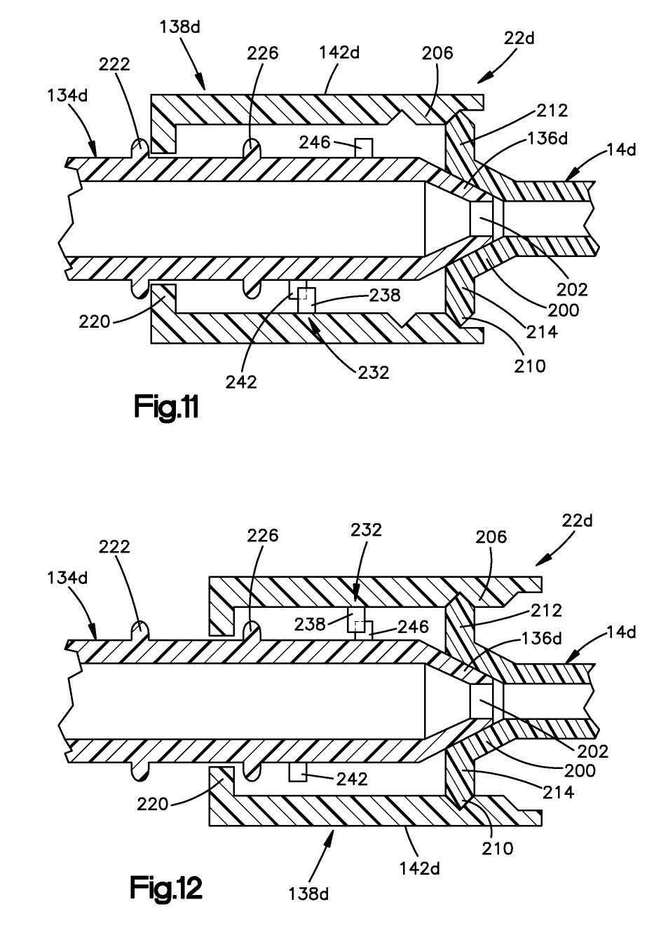

The connector assembly 22d (FIGS. 11 and 12) is utilized to connect a catheter 14d with a tubular body or conduit 134d. When the tubular body or conduit 134d is to be connected with the catheter 14d, a funnel shaped end portion 200 of the catheter 14d is manually moved into telescopic engagement with an axially tapered leading end portion 136d of the tubular body or conduit 134, in the manner illustrated schematically in FIG. 11. At this time, the tubular body or conduit 134d is connected in fluid communication with the catheter 14d through a circular opening 202 in the leading end portion 136d of the tubular body 134d.

When the catheter 14d is initially positioned in engagement with the leading end portion 136d of the tubular body 134d, in the manner illustrated schematically in FIG. 11, the catheter 14d is manually held in place on the leading end portion 136d of the tubular body 134d. The leuer lock nut 138d is then rotated about its longitudinally extending central axis, which is coincident with the longitudinally central axis of the tubular conduit 134d. As the leuer lock nut 134d is rotated, an internal thread 206 on the leuer lock nut 138d engages an external thread 210 on the catheter 14d. The external thread 210 on the catheter 14d may be formed by a pair of flanges 212 and 214 located on diametrically opposite sides of the catheter 14d. Of course, the external thread may be formed on a circular end portion of the catheter 14d if desired.

Once the leading end portion 136d of the tubular body 134d has been telescopically inserted into the funnel shaped end portion 200 of the catheter 14d to form a fluid tight seal between the catheter and tubular body, the leuer lock nut 138d is rotated about its longitudinal central axis. Rotation of the leuer lock nut 138d about its longitudinal central axis causes the internal thread 206 on the leuer lock nut to move along the external thread 210 on the funnel shaped end portion 200 of the catheter 14d. When this occurs, an annular flange 220 on the trailing end of the leuer lock nut moves out of engagement with an annular rim or ring 222 on the tubular body 134d and moves rightward (as viewed in FIG. 11) towards the catheter 14d.

As this occurs, the flange 220 on the trailing end of the leuer lock nut 138 moves toward and into engagement with an annular ring rim 226 on the tubular body 134d, in the manner illustrated schematically in FIG. 12. This results in the catheter 14d being pressed firmly against the leading end portion 136d of the tubular body 134d by the internal thread 206 on the leuer lock nut 138d. At this time, the annular flange 220 on the leuer lock nut is pressed against the ring 226 and the funnel shaped end portion 200 of the catheter 14d is firmly pressed against the leading end portion 136d of the tubular body 134d to maintain a fluid tight seal between the leading end portion 136d of the tubular body 134d and the funnel shaped end portion 200 of the catheter 14d.

When the leuer lock nut 138d has been tightened to firmly interconnect the catheter 14d and the tubular body 134d, the leuer lock nut 138d should be oriented so that flat surface area 142d on the nut faces toward the patient's body, that is, toward the body portion 12 illustrated in FIG. 1. A positioning device 232 is provided in association with the leuer lock 138d and the tubular body 134d to orient the leuer lock nut relative to the tubular body. The positioning device 232 orients the leuer lock nut 138d relative to the tubular body 134d in an orientation such that the flat surface 142d on the leuer lock nut faces downward (as viewed in FIG. 12) toward the patient's body.

The internal thread 236 on the leuer lock nut 138d has a relatively large pitch so that when the leuer lock nut 138d is rotated through one-half of a revolution (180°), the leuer lock nut 138d is moved axially along the tubular body 134d from the position shown in FIG. 11 to the position shown in FIG. 12. The manner in which the internal thread 206 on the leuer lock nut 138d cooperates with the external thread 210 on the catheter 14d is well known and, in the specific embodiment of the invention illustrated in FIGS. 10-12 is, the same as is disclosed in U.S. Pat. No. 3,876,234. The pitch on the internal thread 206 on the leuer lock nut 138d is selected so that when the leuer lock nut is rotated through one-half of a revolution, the nut moves axially from the position illustrated in FIG. 11 to the position illustrated in FIG. 12.

In the illustrated embodiment of the invention, the leuer lock nut 138d is rotated through only one-half of a revolution (180°) to effect a combined axial and rotational movement of the leuer lock nut from the initial position of FIG. 11 to the connected position of FIG. 12. However, the leuer lock nut 138d may be constructed so as to rotate through a greater distance than one-half of a revolution to move from the initial position of FIG. 11 to the connected position of FIG. 12. For example, the leuer lock nut 138d may be constructed so as to rotate through 540° or 900° to move from the initial position of FIG. 11 to the connected position of FIG. 12.

A positioning device 232 is provided in association with the leuer lock nut 138d and the tubular body 134d. The positioning device 232 functions to make certain that the flat surface area 142d on the leuer lock nut is facing toward the patient's body when the catheter 14d is connected with the tubular body 134d by the leuer lock nut. The positioning device 232 is constructed so as to require the lock nut 138d to be in a predetermined orientation when the internal thread 206 on the leuer lock nut 138d initially engages the external thread 210 on the catheter 14d. In the embodiment of the invention illustrated in FIGS. 11 and 12, the predetermined initial orientation of the leuer lock nut 138d is with the flat surface 142d facing upwardly in the manner illustrated in FIGS. 10 and 11.

When the catheter 14d is to be connected with the leuer lock nut 138d, the catheter is manually positioned in engagement with the leading end portion 136d of the tubular body 134d. The leuer lock nut 138d is moved to a preselected initial position (FIG. 11). When the leuer lock nut 138d is in the preselected initial position, the flat side surface 142d on the leuer lock nut will be facing away from the patient's body and will be fully exposed to an individual who is interconnecting the catheter 14d and the tubular body 134d. When the leuer lock nut 138d is in the initial or disconnected position of FIG. 11, a cylindrical positioning pin 238 on the leuer lock nut 138d is disposed in engagement with a cylindrical start or initial pin 242 on the tubular body 134. At this time, the flange 220 is in engagement with the ring 222.

As the leuer lock nut 138d is rotated through one-half of a revolution, that is, 180 degrees, the cooperation between the internal thread 206 on the leuer lock nut and the external thread 210 on the catheter 14d causes the lock nut 138d to move axially from the initial position shown in FIG. 11 to the connected position shown in FIG. 12. As the leuer lock nut is rotated and moved axially, the positioning pin 238 on the leuer lock nut moves into engagement with a cylindrical stop pin 242 disposed on the tubular body 134d. Engagement of the positioning pin 238 on the lock nut 138d with the stop pin 242, in the manner illustrated schematically in FIG. 12, interrupts rotation and axial movement of the leuer lock nut with the flat side surface 142d facing downward (as viewed in FIG. 12) toward the patient's body. At this time, the flange 220 is in engagement with the ring 222.

Although only the lock nut 138d and tubular conduit 144d have been illustrated in FIGS. 10-12, it should be understood that a positioning apparatus, corresponding to the positioning apparatus 10 of FIGS. 1-6, is associated with the tubular body 134d and the lock nut 138d in the manner previously explained. Thus, a flexible base, corresponding to the base 26 of FIGS. 1-6 is secured to the patient's body by strips of adhesive, corresponding to the left and right adhesive strips 38 and 46 of FIG. 3. The positioning apparatus which is associated with the tubular body 134d and leuer lock nut 138d of FIGS. 10-12 has the same construction as the positioning apparatus previously described herein in conjunction with FIGS. 1-6.

### Auxiliary Conduit

In the embodiment of the invention illustrated in FIGS. 1-12, a single fluid conduit, designated by the numeral 16 in FIG. 1, has been associated with the positioning apparatus 10 and catheter 14. In the embodiment of the invention illustrated in FIG. 13, an auxiliary conduit is connected with the main conduit of the apparatus. Since the embodiment of the invention illustrated in FIG. 13 is generally similar to the embodiment of the invention illustrated in FIGS. 1-12, similar numerals will be utilized to designate similar components. The suffix letter “e” being associated with the numerals of FIG. 13 to avoid confusion.

A positioning apparatus 10e is utilized to position a catheter 14e and a fluid conduit 16e relative to a patient's body. The fluid conduit 16e includes a main conduit 250 and an auxiliary conduit 252. The auxiliary conduit 252 is connected in fluid communication with the main conduit 250 at a connection 254. This enables fluid from two different sources to be conducted to the catheter 14e through the fluid conduit 16e. Thus, a first or primary fluid may be conducted through the main conduit 250 to the catheter 14e. A second fluid may be conducted to the catheter 14e through the auxiliary conduit 252 and the portion of the main conduit 250 which is located downstream from the connection 254.

Although only a single auxiliary conduit 252 has been illustrated in FIG. 13, additional auxiliary conduits may be connected to the main conduit 250 if desired. This would enable a plurality of different fluids to be conducted to the main conduit 250 through auxiliary conduits which branch off from the main conduit.

### CONCLUSION

In view of the foregoing description, it is apparent that the present invention provides a new and improved positioning apparatus 10 for use in positioning a catheter 14 or other device relative to a patient's body 12. The apparatus 10 includes a base 26 having a central portion 34 which is to be connected with the catheter 14. In addition, the base 26 has first and second sections 28 and 30 which extend from the central portion 34 of the base. Strips 38 and 46 of adhesive are disposed on the sections 28 and 30 of the base. Separator strips 58 and 70 engage the strips 38 and 46 of adhesive when the separator strips are in a blocking condition. The separator strips 58 and 70 are movable to a disengaged condition in which they are separated from the strips 38 and 46 of adhesive to enable the strips of adhesive to adhere to the patient's body. Absorbent pads 62 and 74 are disposed on the separator strips 58 and 70 and face away from the adhesive to facilitate absorption of any liquid disposed on the patient's body at a location to be engaged by a strip 38 or 46 of adhesive.

A connector assembly 10 is provided to interconnect the catheter 14 and a conduit 16. The connector assembly includes a nut 138 which engages the catheter 14. A positioning device 232 may be utilized to orient the nut 138 so that a pre-selected surface area 142 on the nut faces toward the patient's body.

The present invention has a plurality of features which are disclosed herein as being utilized together. However, it is contemplated that each of the features may be used separately from other features of the invention. It is also contemplated that each of the features of the invention may be utilized in combination with features from the prior art. For example, the positioning apparatus of FIGS. 1-6 may be utilized with or without the positioning device 232 of FIGS. 10-12. As a further example, the fluid conduit 16 of FIG. 1 may be utilized either with or without the auxiliary fluid conduit 252 of FIG. 13.

Having described the invention, the following is claimed:

1. A positioning apparatus for use in positioning a catheter relative to a patient's body, said apparatus comprising a base having a central portion to be connected with the catheter, said base having a first section which extends in a first direction from the central portion of said base and a second section which extends in a second direction from the central portion of said base, a first strip of adhesive disposed on said first section of said base, a second strip of adhesive disposed on said second section of said base, a first separator strip which engages said first strip of adhesive, said first separator strip being movable from a blocking condition extending across said first strip of adhesive to a disengaged condition in which said first separator strip is disengaged from said first strip of adhesive to enable said first strip of adhesive to adhere to the patient's body, a second separator strip which engages said second strip of adhesive, said second separator strip being movable from a blocking condition extending across said second strip of adhesive to a disengaged condition in which said second separator strip is disengaged from said second strip of adhesive to enable said second strip of adhesive to adhere to the patient's body, a first absorbent pad disposed on said first separator strip and facing away from said first strip of adhesive to facilitate absorption of any liquid disposed on the patient's body at a location to be engaged by said first strip of adhesive, said first absorbent pad being movable with said first separator strip relative to said first strip of adhesive upon movement of said first separator strip from the blocking condition to the disengaged condition, and a second absorbent pad disposed on said second separator strip and facing away from said second strip of adhesive to facilitate absorption of any liquid disposed on the patient's body at a location to be engaged by said second strip of adhesive, said second absorbent pad being movable with said second separator strip relative to said second strip of adhesive upon movement of said second separator strip from the blocking condition to the disengaged condition.

2. An apparatus as set forth in claim 1 wherein said base is flexible to enable said first and second sections of said base to be moved relative to each other and relative to said central portion of said base.

3. An apparatus as set forth in claim 1 further including a connector assembly mounted on said central portion of said base to connect the catheter with a conduit, said connector assembly including a nut which is rotatable relative to said central portion of said base, said nut having a flat side surface with an extent in a direction perpendicular to a central axis of said nut less than a maximum cross sectional area of said nut as measured in a plane parallel to said flat side surface and containing the central axis of said nut.

4. An apparatus as set forth in claim 1 further including a connector assembly mounted on said central portion of said base to connect the catheter with a conduit, said connector assembly including a nut which is rotatable relative to said central portion of said base a positioning device which orients the nut so that a pre-selected surface area on the nut faces toward the patient's body when the catheter is connected with the conduit.

5. An apparatus as set forth in claim 1 wherein said first section of said base, said first separator strip and said first absorbent pad are movable between a retracted condition in which they are folded at a first plurality of fold lines and an extended condition in which they are unfolded at the first plurality of fold lines, said second section of said base, said second separator strip and said second absorbent pad are movable between a retracted condition in which they are folded at a second plurality of fold lines and an extended condition in which they are unfolded at the second plurality of fold lines.

6. An apparatus as set forth in claim 1 wherein said first separator strip has a first surface area which faces away from said first strip of adhesive, a second surface area which is disposed in engagement with said first surface area when said first separator strip is in the blocking condition, and a first fold portion which interconnects said first and second surface areas on said first separator strip and is disposed adjacent to said central portion of said base when said first separator strip is in the blocking condition, said second separator strip has a first surface area which faces away from said second strip of adhesive, said second separator strip having a second surface area which is disposed in engagement with said first surface area on said second separator strip when said second separator strip is in the blocking condition, and a second fold portion which interconnects said first and second surface areas on said second separator strip and is disposed adjacent to said central portion of said base when said second separator strip is in the blocking condition.

7. An apparatus as set forth in claim 1 further including a pad connected with said central portion of said base and engageable with the patient's body when said first and second separator strips are in their disengaged conditions.

8. An apparatus as set forth in claim 1 further including a first portion of a fastener connected with said central portion of said base and a second portion of said fastener connected with an end portion of a strip of material, said second portion of said fastener being engageable with said first portion of said fastener to connect the end portion of said strip of material with said base to facilitate wrapping said strip of material around a portion of the patient's body.

9. An apparatus as set forth in claim 1 wherein said first section of said base has a longitudinal central axis which is skewed at an acute angle to a longitudinal central axis of the catheter, said second section of said base has a longitudinal central axis which is skewed at an acute angle to the longitudinal central axis of the catheter.

10. An apparatus as set forth in claim 8 further including a connector assembly mounted on said central portion of said base to connect the catheter with a conduit, said connector assembly including a nut which is rotatable about the longitudinal central axis of the catheter, said nut being disposed between and spaced from said first and second sections of said base.

11. An apparatus as set forth in claim 10 wherein said nut has a flat side surface which faces toward the patient's body when the catheter is connected with the conduit.

12. A positioning apparatus for use in positioning a catheter relative to a patient's body, said apparatus comprising a base having a central portion to be connected with the catheter, said base having a first section which extends in a first direction from the central portion of said base and a second section which extends in a second direction from the central portion of said base, a first strip of adhesive disposed on said first section of said base to connect the first section of the base with the patient's body, a second strip of adhesive disposed on said second section of said base to connect the second section of the base with the patient's body, a connector assembly mounted on said central portion of said base to connect the catheter with a conduit, said connector assembly including a nut which is rotatable relative to said central portion of said base and a positioning device which orients the nut so that a flat surface area on the nut faces toward the patient's body when the catheter is connected with the conduit.

13. An apparatus as set forth in claim 12 further including a first separator strip which engages said first strip of adhesive, said first separator strip being movable from a blocking condition extending across said first strip of adhesive to a disengaged condition in which said first separator strip is disengaged from said first strip of adhesive to enable said first strip of adhesive to adhere to the patient's body, a second separator strip which engages said second strip of adhesive, said second separator strip being movable from a blocking condition extending across said second strip of adhesive to a disengaged condition in which said second separator strip is disengaged from said second strip of adhesive to enable said second strip of adhesive to adhere to the patient's body, a first absorbent pad disposed on said first separator strip and facing away from said first strip of adhesive to facilitate absorption of any liquid disposed on the patient's body at a location to be engaged by said first strip of adhesive, said first absorbent pad being movable with said first separator strip relative to said first strip of adhesive upon movement of said first separator strip from the blocking condition to the disengaged condition, and a second absorbent pad disposed on said second separator strip and facing away from said second strip of adhesive to facilitate absorption of any liquid disposed on the patient's body at a location to be engaged by said second strip of adhesive, said second absorbent pad being movable with said second separator strip relative to said second strip of adhesive upon movement of said second separator strip from the blocking condition to the disengaged condition.

14. An apparatus as set forth in claim 13 wherein said base is flexible to enable said first and second sections of said base to be moved relative to each other and relative to said central portion of said base.

15. An apparatus as set forth in claim 12 wherein said positioning device includes a projection on said nut which engages a first projection connected with said base to orient the nut relative to said base prior to connection of the catheter with the conduit, said projection on said nut engages a second projection connected with said base to orient the nut relative to said base when the catheter is connected with the conduit.
